(19) United States
(12) Patent Application Publication
(10) Pub. No.: US 2023/0197265 A1
(43) Pub. Date: Jun. 22, 2023

(54) TECHNIQUES FOR PROVIDING INSIGHTS ACCORDING TO APPLICATION DATA AND BIOMETRIC DATA

(71) Applicant: Oura Health Oy, Oulu (FI)

(72) Inventors: Johanna Leena Kyllikki Still, Oulu (FI); Alec Alexander Singleton, Toronto (CA); Dmitry Sergeev, Helsinki (FI); Jason Russell, Hamilton, MA (US)

(21) Appl. No.: 18/145,640

(22) Filed: Dec. 22, 2022

Related U.S. Application Data
(60) Provisional application No. 63/293,064, filed on Dec. 22, 2021.

(51) Int. Cl. G16H 40/63

(52) U.S. Cl. CPC G16H 40/63

(57) ABSTRACT

Methods, systems, and devices for content delivery are described. A device may receive biometric data associated with a user from a wearable device, and receive application data associated with the user from the device. The application data may be indicative of a previous activity in which the user engaged or an upcoming activity in which the user is to engage. The device may determine content for recommending to the user based on correlating the received biometric data and the received application data. The content may correspond to a respective effectiveness for regulating the received biometric data. In some examples, the content may facilitate improvements to the user’s health and wellness. The device may cause a graphical user interface (GUI) of the device running an application to display the content for recommending to the user based on correlating the received biometric data and the received application data.

### CROSS REFERENCE

The present Application for Patent claims the benefit of U.S. Provisional Pat. Application No. 63/293,064 by Still et al., entitled “TECHNIQUES FOR PROVIDING INSIGHTS ACCORDING TO APPLICATION DATA AND BIOMETRIC DATA,” filed Dec. 22, 2021, assigned to the assignee hereof, and expressly incorporated by reference herein.

### FIELD OF TECHNOLOGY

The following relates to wearable devices and data processing, including techniques for providing insights according to application data and biometric data (also referred to as physiological data).

### BACKGROUND

Some devices may be configured to collect personal data from users. For example, a device may include one or more sensors that collect biometric data (also referred to as physiological data) from a user. Some devices may also be able to perform various actions, such as providing certain insights to the user based at least in part on the collected biometric data. In the example of health and wellness, these devices may provide health and wellness insights using the collected biometric data. However, improved techniques for providing insights to a user may be desired.

### BRIEF DESCRIPTION OF THE DRAWINGS

FIG. 1 illustrates an example of a system that supports techniques for providing insights according to application data and biometric data in accordance with aspects of the present disclosure.

FIG. 2 illustrates an example of a system that supports techniques for providing insights according to application data and biometric data in accordance with aspects of the present disclosure.

FIG. 3 illustrates an example of a graphical user interface (GUI) that supports techniques for providing insights according to application data and biometric data in accordance with aspects of the present disclosure.

FIG. 4 shows a block diagram of an apparatus that supports techniques for providing insights according to application data and biometric data in accordance with aspects of the present disclosure.

FIG. 5 shows a block diagram of a wearable application that supports techniques for providing insights according to application data and biometric data in accordance with aspects of the present disclosure.

FIG. 6 shows a diagram of a system including a device that supports techniques for providing insights according to application data and biometric data in accordance with aspects of the present disclosure.

FIGS. 7 through 9 show flowcharts illustrating methods that support techniques for providing insights according to application data and biometric data in accordance with aspects of the present disclosure.

### DETAILED DESCRIPTION

Various applications may collect biometric data associated with a user to provide insights relevant to the user. In some cases, an application associated with health and wellness may collect and track biometric data, and the like. For example, a health and wellness application may include information associated with a user’s biometric history, and the like. In some other cases, a health and wellness application may be configured to provide various insights and recommendations in the form messages, audio content, video content, etc. to a user based on the user’s biometric history. For example, a health and wellness application may provide various guidance information (e.g., routines, actions, or the like) to improve the health and wellness of the user. Some applications for providing insights to a user may fail to provide the most effective insights in improving the health and wellness of the user. As such, improvements to these health and wellness applications for providing insights that are intended to have a health and wellness impact on a user are needed.

A system including a wearable device and a user device may collect biometric data (also referred to as physiological data) and, based on the collected biometric data, may provide insights to the user. The wearable device (e.g., a wearable ring) may receive biometric data associated with the user. The biometric data may include heart rate data associated with the user, respiratory rate data associated with the user, sleep data associated with the user, activity data associated with the user, or any combination thereof. In some implementations, collected or received biometric data associated with the user may be used, for example, by the user device to determine insights for the user. Additionally, or alternatively, the user device may receive application data associated with the user. The application data may be indicative of a previous activity in which the user engaged (e.g., consumption of caffeine) or an upcoming activity (e.g., a work trip) in which the user is scheduled to engage. In some implementations, collected or received application data may be used, for example, by the user device to determine insights and content for recommending to the user. In other implementations, the user device may determine content for recommending to the user, for example, based on both of collected biometric data and received application data.

Content recommended to the user may correspond to a respective effectiveness (e.g., to provide a desired result) for regulating (e.g., increasing, decreasing, managing, or any combination thereof) the received biometric data. Put another way, a recommended content may provide (e.g., enable, support) a desired result for regulating (e.g., increasing, decreasing, maintaining) the user’s biometrics. For example, the user device may determine that the user has recurring evening meetings on certain days of the week (e.g., Tuesday) and that the user’s biometric data is suboptimal (e.g., below a threshold) on a following day of the week (e.g., Wednesday morning). These evening meetings may thereby have an impact on the user’s sleep schedule. For example, the user may have a sleep issue, such as sleep latency (i.e., a timeframe in which the user typically transitions to a sleep state). Based on the determination, the user device may determine (e.g., select) insights and content that may provide guidance to the user on recovering from the meetings better. In some implementations, as described herein, this may include the user device autonomously or semi-autonomously scheduling an event (e.g., a calendar event) to facilitate improving or managing the user’s health and wellness (e.g., a Readiness Score, a Sleep Score, or the like). For example, the scheduled event may indicate a recommended sleep time for the user to go to bed based on the sleep issue of the user or a recommendation indicating one or more instructions for the user to increase the Readiness Score, Sleep Score, or the like of the user.

As a result, the system facilitates improvements to the user’s biometrics by providing health and wellness insights, such as recommendations based on the user’s biometric data and application data. While much of the present disclosure is described in the context of biometrics, this is not to be regarded as a limitation of the present disclosure. In particular, techniques described herein may enable health and wellness insights for a user, that may help improve the biometrics. Moreover, biometric data and/or application data associated with a user may be used to update any score, measure, metric, or other abstraction associated with a user’s health or activity.

Aspects of the disclosure are initially described in the context of systems supporting physiological data collection from users via wearable devices. Additional aspects of the disclosure are described in the context of an example GUI. Aspects of the disclosure are further illustrated by and described with reference to apparatus diagrams, system diagrams, and flowcharts that relate to techniques for providing insights according to application data and biometric data.

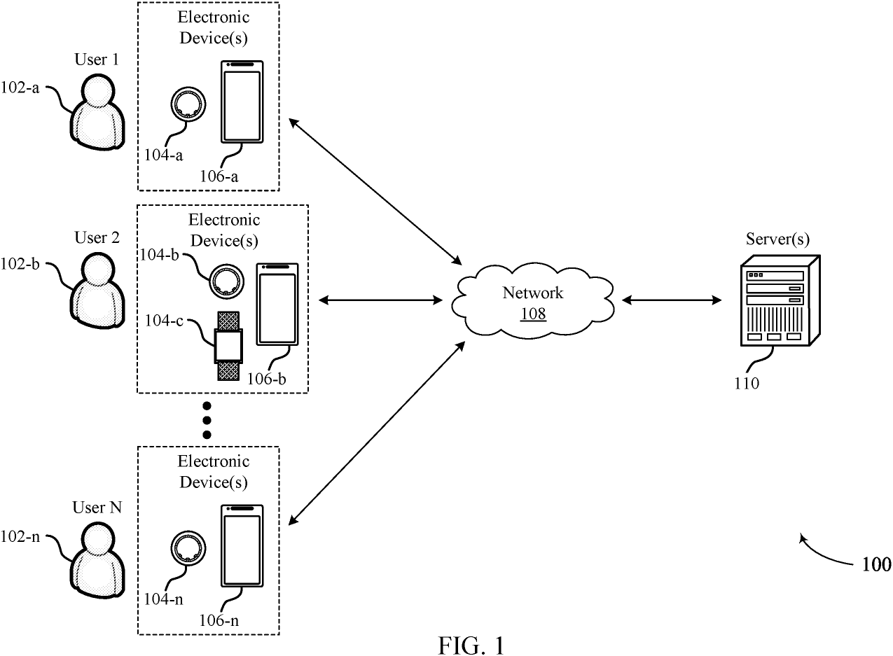

FIG. 1 illustrates an example of a system 100 that supports techniques for providing insights according to application data and biometric data in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user’s 102 finger, a wrist computing device (e.g., a smart watch, fitness band, or bracelet) configured to be worn on a user’s 102 wrist, and/or a head-mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user’s finger. In particular, a ring 104 may utilize one or more LEDs (e.g., red LEDs, green LEDs) that emit light on the palm side of a user’s finger to collect physiological data based on arterial blood flow within the user’s finger. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof. In general, the terms light-emitting components, light-emitting elements, and like terms, may include, but are not limited to, LEDs, micro LEDs, mini LEDs, laser diodes (LDs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time during which a user 102 is asleep, and classify periods of time during which the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time during which the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user’s sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual’s sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user’s natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual’s baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for providing insights according to application data and biometric data. In particular, the system 100 illustrated in FIG. 1 may support techniques for providing insights to a user 102 by causing a user device 106 corresponding to the user 102 to display content for recommending to the user 102. For example, as shown in FIG. 1, User 1 (e.g., a user 102-a) may be associated with a wearable device 104-a (e.g., a ring 104-a) and a user device 106-a. The wearable device 104-a (e.g., a ring 104-a) may receive biometric data (also referred to as physiological data) associated with the User 1 (e.g., the user 102-a). The biometric data may include heart rate data associated with the User 1 (e.g., the user 102-a), respiratory rate data associated with the User 1 (e.g., the user 102-a), sleep data associated with the User 1 (e.g., the user 102-a), activity data associated with the User 1 (e.g., the user 102-a), or any combination thereof.

In some implementations, collected or received biometric data associated with the User 1 (e.g., the user 102-a) may be used, for example, by the user device 106 (e.g., the user device 106-a) associated with the User 1 (e.g., the user 102-a) to determine content for recommending to the User 1 (e.g., the user 102-a). In some other implementations, any of the components of the system 100, including the wearable device 104-a (e.g., a ring 104-a), the user device 106-a associated with User 1 (e.g., a user 102-a), the one or more servers 110, or any combination thereof, may determine content for recommending to the User 1 (e.g., the user 102-a), for example, based on collected or received biometric data associated with the User 1 (e.g., the user 102-a).

Additionally, or alternatively, in the system 100, the user device 106-a may receive application data associated with the User 1 (e.g., the user 102-a). The application data may be indicative of a previous activity in which the User 1 (e.g., the user 102-a) engaged or an upcoming activity in which the User 1 (e.g., the user 102-a) is to engage. An application running fully or partially (e.g., in the case of a cloud application) on the user device 106-a may be a lifestyle application (e.g., fitness, food, a journal, travel, period tracking, or the like), a social media application, a utility application (e.g., reminders, calculator, weather, or the like), an entertainment application (e.g., multimedia streaming, gaming, or the like), a productivity application (e.g., docs, sheets, files, or the like), an information outlet application (e.g. news, blogs, or the like), or any combination thereof.

In some implementations, collected or received application data associated with the User 1 (e.g., the user 102-a) may be used, for example, by the user device 106 (e.g., the user device 106-a) associated with the User 1 (e.g., the user 102-a) to determine content for recommending to the User 1 (e.g., the user 102-a). In some other implementations, any of the components of the system 100, including the wearable device 104-a (e.g., a ring 104-a), the user device 106-a associated with User 1 (e.g., a user 102-a), the one or more servers 110, or any combination thereof, may determine content for recommending to the User 1 (e.g., the user 102-a), for example, based on collected or received application data associated with the User 1 (e.g., the user 102-a). In other implementations, any of the components of the system 100, including the wearable device 104-a (e.g., a ring 104-a), the user device 106-a associated with User 1 (e.g., a user 102-a), the one or more servers 110, or any combination thereof, may determine content for recommending to the User 1 (e.g., the user 102-a), for example, based on both (e.g., a combination) of collected biometric data and received application.

Content recommended to User 1 (e.g., the user 102-a) may correspond to a respective effectiveness for regulating (e.g., increasing, decreasing, managing, or any combination thereof) the received biometric data. For example, any of the components of the system 100, including the wearable device 104-a (e.g., a ring 104-a), the user device 106-a associated with User 1 (e.g., a user 102-a), the one or more servers 110, or any combination thereof, may determine that the User 1 (e.g., a user 102-a) has recurring evening meetings on certain days of week (e.g., Wednesday and Thursday) and that the User 1 (e.g., a user 102-a) biometric data is bad (e.g., below a threshold) on following days of the week (e.g., Friday morning). These evening meetings may have an impact on the User 1 (e.g., a user 102-a) sleep schedule. For example, the User 1 (e.g., a user 102-a) may have a sleep issue, such as sleep latency (i.e., a timeframe in which the User 1 (e.g., a user 102-a) typically transitions to a sleep state, such as a rapid eye movement (REM) sleep state or a non-REM sleep state. In other examples, content recommended to User 1 (e.g., the user 102-a) may correspond to the respective effectiveness for regulating (e.g., increasing, decreasing, managing, or any combination thereof) the received biometric data may be related to daytime heart rate. For example, any of the components of the system 100, including the wearable device 104-a (e.g., a ring 104-a), the user device 106-a associated with User 1 (e.g., a user 102-a), the one or more servers 110, or any combination thereof, may determine hear rate elevations at certain times of the week that corresponds to recurring meetings.

Based on the determination, any of the components of the system 100, including the wearable device 104-a (e.g., a ring 104-a), the user device 106-a associated with User 1 (e.g., a user 102-a), the one or more servers 110, or any combination thereof, may determine (e.g., select) content that may provide guidance to the User 1 (e.g., a user 102-a) on recovering from the meetings better. In the system 100, the user device 106-a associated with the User 1 (e.g., a user 102-a) may cause a GUI of the user device 106-a running an application to display content for recommending to the User 1 (e.g., a user 102-a). In some examples, the content may include a user-specific notification indicating a Readiness Score (e.g., impacted based on the sleep issue of the User 1 (e.g., a user 102-a)) or a recommendation indicating one or more instructions for the User 1 (e.g., a user 102-a) to increase the Readiness Score of the User 1 (e.g., a user 102-a).

While the sleep latency serves as an example of biometric data and the evening meetings serve as an example of application data, various other forms of biometric data and application data may be used to recommend content to the User 1 (e.g., a user 102-a). For example, in the system 100, any of the components of the system 100, including the wearable device 104-a (e.g., a ring 104-a), the user device 106-a associated with User 1 (e.g., a user 102-a), the one or more servers 110, or any combination thereof, may determine a pattern between the received biometric data and the received application data for content recommendation. For example, in the system 100, any of the components of the system 100, including the wearable device 104-a (e.g., a ring 104-a), the user device 106-a associated with User 1 (e.g., a user 102-a), the one or more servers 110, or any combination thereof, may determine a recurrency of an activity (e.g., meetings, lunches, exercises, appointments, trips, or the like) over a set of occasions (e.g., days, weeks, months). Any of the components of the system 100, including the wearable device 104-a (e.g., a ring 104-a), the user device 106-a associated with User 1 (e.g., a user 102-a), the one or more servers 110, or any combination thereof, may determine content that may provide guidance to the User 1 (e.g., a user 102-a) on improving or managing the health and wellness of the User 1 (e.g., a user 102-a) based on recurrency of the activity.

In some implementations, in the system 100, any of the components of the system 100, including the wearable device 104-a (e.g., a ring 104-a), the user device 106-a associated with User 1 (e.g., a user 102-a), the one or more servers 110, or any combination thereof, may determine that the user’s activities are scheduled for early afternoon and their evening is free, corresponding with good scores (e.g., a Sleep Score) on the following morning, the system 100 may highlight this and congratulate the user on good scheduling via a GUI of the user device.

Accordingly, in the system 100, any of the components of the system 100, including the wearable device 104-a (e.g., a ring 104-a), the user device 106-a associated with User 1 (e.g., a user 102-a), the one or more servers 110, or any combination thereof, may access application data associated with a user 102 and evaluate the application data against other data, such as biometric data collected by a wearable device 104 (e.g., a ring 104) to provide personalized insights for improving or managing the health and wellness of the user 102. That is, the system 100 may provide the personalized insights in the form of a personalized notification via the wearable device 104 or the user device 106.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

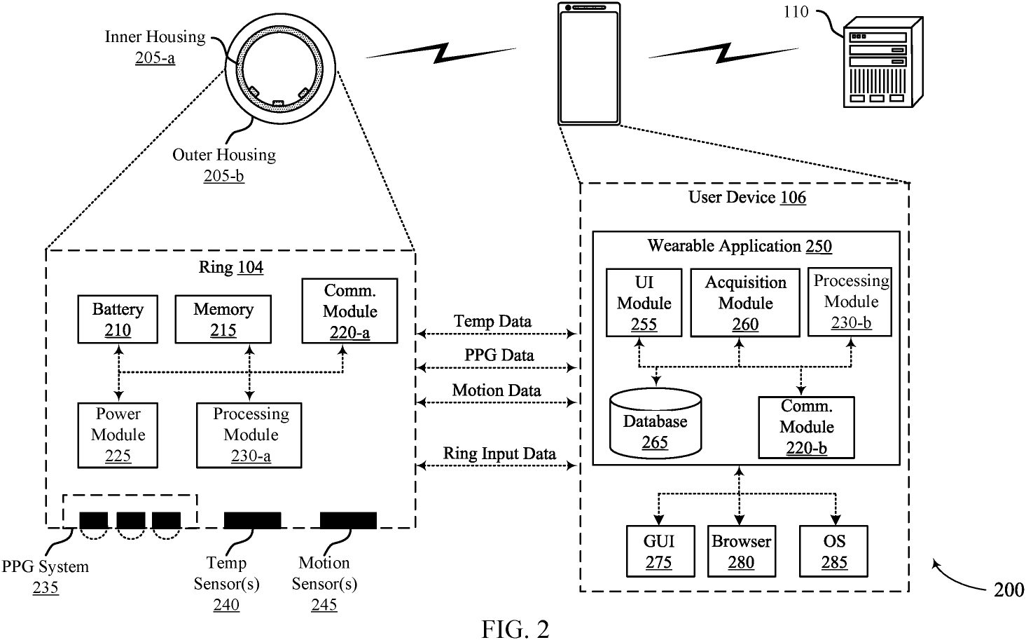

FIG. 2 illustrates an example of a system 200 that supports techniques for providing insights according to application data and biometric data in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user’s finger, and may determine one or more user physiological parameters when worn around the user’s finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user’s finger (e.g., using a clamp, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user’s finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user’s finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user’s finger (e.g., skin temperature). In some implementations, the temperature sensor 240 may contact the user’s skin. In other implementations, a portion of the housing 205 (e.g., the inner housing 205-a) may form a barrier (e.g., a thin, thermally conductive barrier) between the temperature sensor 240 and the user’s skin. In some implementations, portions of the ring 104 configured to contact the user’s finger may have thermally conductive portions and thermally insulative portions. The thermally conductive portions may conduct heat from the user’s finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user’s temperature over time. For example, the processing module 230-a may sample the user’s temperature according to a sampling rate. An example sampling rate may include one sample per second, although the processing module 230-a may be configured to sample the temperature signal at other sampling rates that are higher or lower than one sample per second. In some implementations, the processing module 230-a may sample the user’s temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user’s finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user’s finger (e.g., any finger). For example, one or more temperature sensors 240 on the ring 104 may acquire a user’s temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user’s finger may differ from the temperature measured at a user’s wrist or other external body location. Additionally, the distal temperature measured at a user’s finger (e.g., a “shell” temperature) may differ from the user’s core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user’s finger. The PPG signal generated by the PPG system 235 may indicate the perfusion of blood in the illuminated region. For example, the PPG signal may indicate blood volume changes in the illuminated region caused by a user’s pulse pressure. The processing module 230-a may sample the PPG signal and determine a user’s pulse waveform based on the PPG signal. The processing module 230-a may determine a variety of physiological parameters based on the user’s pulse waveform, such as a user’s respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user’s finger. In some implementations, the PPG system 235 may be configured as a transmissive PPG system 235 in which the optical transmitter(s) and optical receiver(s) are arranged opposite to one another, such that light is transmitted directly through a portion of the user’s finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user’s heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user’s respiratory rate over time. For example, the processing module 230-a may determine respiratory rate based on frequency modulation, amplitude modulation, or baseline modulation of the user’s IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user’s finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user’s physiological parameters may be measured by sensors included on a ring 104, other devices may measure a user’s physiological parameters. For example, although a user’s temperature may be measured by a temperature sensor 240 included in a ring 104, other devices may measure a user’s temperature. In some examples, other wearable devices (e.g., wrist devices) may include sensors that measure user physiological parameters. Additionally, medical devices, such as external medical devices (e.g., wearable medical devices) and/or implantable medical devices, may measure a user’s physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS) 285, a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user’s sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time in which the respective users typically sleep.

In some implementations, each overall score for a user for each respective day (e.g., Sleep Score, Readiness Score) may be determined/calculated based on one or more “contributors,” “factors,” or “contributing factors.” For example, a user’s overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user’s sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user’s overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user’s needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user’s sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user’s resting heart rate to stabilize during the night. A sign of a very good recovery is that the user’s resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user’s highest temperature during the nap is at least 0.5° C. higher than the highest temperature during the longest period. In some aspects, the ring may measure a user’s body temperature while the user is asleep, and the system 200 may display the user’s average temperature relative to the user’s baseline temperature. If a user’s body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for providing insights according to application data and biometric data. In particular, the system 200 illustrated in FIG. 2 may support techniques for providing insights to a user by causing a user device 106 corresponding to the user to display, via the GUI 275, content for recommending to the user. The ring 104 may receive biometric data of a respective user associated with the ring 104. The biometric data may include heart rate data, respiratory rate data, sleep data, activity data, or any combination thereof. In some implementations, collected or received biometric data of a respective user associated with the ring 104 may be used, for example, by the user device 106 associated with the user to determine content for recommending to the user.

Additionally, or alternatively, in the system 200, the user device 106 may receive application data associated with the respective user. The application data may be indicative of a previous activity in which the respective user engaged or an upcoming activity in which the respective user will engage. The application data may include event information (e.g., activity information) indicating an event the user is to engage in, timing information indicating a duration of the event associated with the user, location information indicating a location of the event associated with the user, or any combination thereof. In some implementations, collected or received application data of the user associated with the ring 104 may be used, for example, by the user device 106 associated with the user to determine content for recommending to the user. In other implementations, collected biometric data by the ring 104 and received application data by the user device 106 associated with the user may be used, for example, by the user device 106 to determine content for recommending to the user.

As noted previously, content recommended to a respective user may correspond to a respective effectiveness for regulating (e.g., increasing, decreasing, managing, or any combination thereof) the received biometric data. In the system 200, the acquisition module 260 of the user device 106 may acquire biometric data and/or application data associated with a user and forward the biometric data and/or the application data to the processing module 230-b to determine whether the biometric data satisfies a threshold and/or determine a previous activity in which the user engaged or an upcoming activity in which the user will engage. For example, the user device 106 (e.g., based on the processing module 230-b) may determine that the user is experiencing high stress based on one or more values of the biometric data (e.g., a resting heart rate above a baseline threshold for the user, or the like). The system 200 may thus provide (e.g., recommend in the form of a notification or other types of messages) insights (e.g., content) to improve the user’s health and wellness (e.g., reduce the user’s stress).

In some implementations, the user device 106 (e.g., based on the processing module 230-b) may schedule at least one event based on correlating the received biometric data and the received application data associated with the user. The at least one event may include event information indicating an activity the user is to engage in for regulating the received biometric data, timing information indicating a duration of the activity the user is to engage in for regulating the received biometric data, location information indicating a location of the activity the user is to engage in for regulating the received biometric data, or any combination thereof.

In the example case of high stress, the user device may schedule at least one calendar event defined within an application associated with the ring 104 and the user device 106. The calendar event may indicate an activity (e.g., meditate, go for a walk, or the like) for the user to improve the user’s well-being. For example, the user device 106 (e.g., based on the processing module 230-b) may schedule at least one calendar event, that may be a scheduled exercise, a scheduled time for the exercise, a duration of the exercise, a location of the exercise (e.g., a current location of the user, or nearby recreation facility or park, or the like), or other activity for reducing the user’s stress level.

A user may accept or reject an event that is scheduled (e.g., added, recommended, or the like) autonomously or semi-autonomously by the user device 106. For example, the user device 106 may cause the GUI 275 of the user device 106 to prompt the user for feedback based on the at least one scheduled calendar event. The feedback may include an acceptance of the at least one calendar event or a rejection of the at least one calendar event by the user. In some implementations, the feedback may be selected from a set of feedback responses displayed via the GUI 275 with the prompt. Based on the feedback from the user, in some examples, the user device 106 may keep the scheduled event. However, in some other examples, based on the feedback, the user device may remove the at least one calendar event defined within the application associated with the ring 104 and the user device 106. In this example, the user device 106 may adjust one or more parameters of a machine learning model for improving content recommendations (e.g., scheduling of events to improve the health and wellness of the user). The machine learning model may be trained (e.g. iteratively or non-iteratively) to identify content for regulating the received biometric data of the user based on the received application data.

In some implementations, in the system 200, the acquisition module 260 of the user device 106 may acquire application data associated with a user and forward the application data to the processing module 230-b to identify an upcoming activity in which the user is to engage. For example, in the context of calendar events, the user device 106 may identify at least one calendar event based on the received application data. In this example, the calendar event may be an upcoming trip for the user. As such, the system 200 (e.g., the user device 106) may provide personalized recommendations (e.g., sleep time, or the like) to prepare the user for the upcoming trip (e.g., reduce jet lag). In another example, the calendar even may be a training event (e.g., a marathon), such that the system 200 (e.g., the user device 106) may provide personalized recommendations (e.g., training timing, recovery duration, sleep duration, or the like) to prepare the user for the upcoming training event (e.g., improve training performance).

The system 200 may process additional data for providing insights to a user for improving or managing the health and wellness of the user. For example, the system 200 may collect sensor data from one or both of the ring 104 or the user device 106. The sensor data may include sound data associated with a physical environment. The user device 106 may process the sound data, for example, via the processing module 230-b to determine whether ambient noise levels are above or below threshold levels, that may prompt sleep issues for the user, and thereby a Readiness Score for the user. In this example, the content the user device 106 displays via the GUI 275 may include a recommendation indicating one or more instructions for the user to reduce a noise level during a duration (e.g., at night) to maintain a threshold Readiness Score of the user (e.g., a minimum Readiness Score of the user, a predetermined Readiness Score of the user, or the like).

Other examples of sensor data may include temperature data associated with the physical environment, as well as light data (e.g., collected by a light sensor, or the like). In these examples, the user device 106 may process the temperature data and/or light data, for example, via the processing module 230-b to determine whether an ambient temperature or light level associated with the physical environment has an impact on the user’s health and wellness. Based on the determination, the content the user device 106 displays via the GUI 275 may include a recommendation indicating one or more instructions for the user to adjust (e.g., increase or decrease) the ambient temperature or light level in the physical environment. The user device 106 may also analyze other data from other applications running on the user device 106, such as a weather application to determine insights (e.g., recommended ambient room temperature for the user) based on current or future weather and respective temperatures.

In some implementations, the system 200 may process additional data from other applications associated with the user device 106 and/or data from a community of users for providing insights to a user for improving or managing the health and wellness of the user. For example, the system 200 may analyze aggregated data from the community of users for providing insights to a user for improving or managing the health and wellness of the user. In some implementations, the system 200 may determine a pattern based on aggregated data from the community of users for providing insights to the user for improving or managing the health and wellness of the user. Examples of patterns may include sleep schedules, exercises, use of certain applications, nutrition, or the like. For example, the system 200 may determine a pattern based on performing image pattern recognition on images or the like posted on a social media application by a user or a community of users, etc. to provide insights to the user for improving or managing the health and wellness of the user. By way of example, the system 200 may determine from one or more social media posts associated with a user a certain activity in which the user engaged (e.g., that the user consumes certain beverages and/or foods). The system 200 may also analyze timestamps associated with the one or more media posts to provide insights to the user about the activity and the impact on the user’s health and wellness. For example, the system 200 may determine that the user consumes alcohol at night or has a late meal, that may impact the user’s sleep schedule. The system 200 may determine this insight based on correlating the user’s collected biometric data to the activity in which the user engaged. In some examples, the system 200 may analyze application data across various applications via an application programming interface (API), that allows multiple applications to communicate and exchange information (e.g., user data) with each other.

The user device 106 may receive user data associated with the user from one or more applications associated with the ring 104 or the user device 160, or any combination thereof. The one or more applications may include a lifestyle application, a social media application, a utility application, an entertainment application, a productivity application, an information outlet application, or any combination thereof. The user device 106 may process the user data via the processing module 230-b by correlating the received user data with the received biometric data and the received application data to determine content for recommending to the user. For example, the content (e.g., activities or multimedia content) may be recommend to the user based on inferences made from the user’s application data on one or more of the example applications or from groups of users.

Accordingly, in the system 200, any of the components of the system 200, including the ring 104 or the user device 106, or any combination thereof, may access application data associated with a user and evaluate the application data against other data, such as biometric data collected by the ring 104 and/or other applications as described herein, to provide personalized insights for improving or managing the health and wellness of the user. That is, the system 100 may provide the personalized insights in the form of a personalized notification via the ring 104 and/or the user device 106.

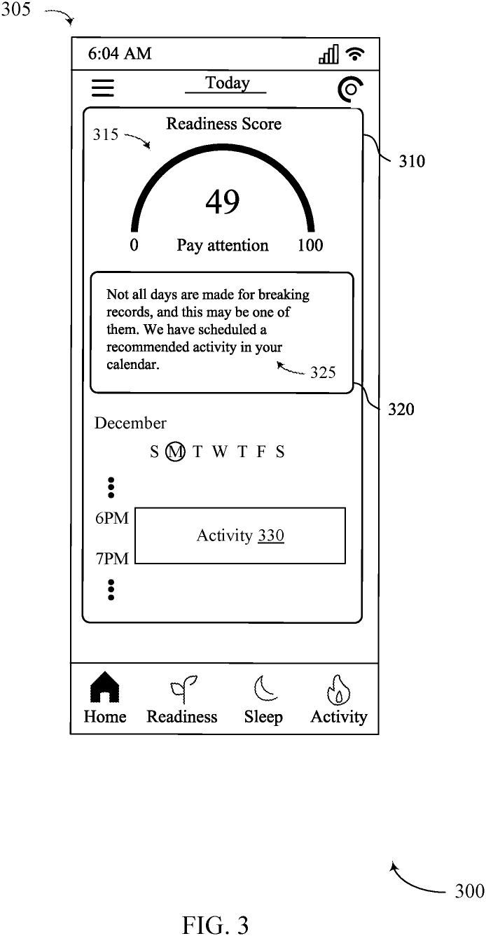

FIG. 3 illustrates an example of a GUI 300 that supports techniques for providing insights according to application data and biometric data in accordance with aspects of the present disclosure. The GUI 300 may implement, or be implemented by, aspects of the system 100 or the system 200, or any combination thereof. In some examples, the GUI 300 may be an example of a GUI of a user device, that may be examples of GUIs and user devices as described with reference to FIGS. 1 and 2. For example, the GUI 300 may be an example of a GUI 275 of a user device 106 as described with reference to FIGS. 1 and 2. In the example of FIG. 3, the GUI 300 may include an application interface 305, that may be displayed to a user 102 via the GUI 300.

The application interface 305 may be associated with an application running on a user device 106. In some examples, the application interface 305 may include a set of graphical elements (also referred to as widgets or components) that the application provides so that a user 102 may provide input to, and receive output from, the application via the application interface 305. In some examples, one or more operations associated with the GUI 300 may be performed based on a manipulation of the one or more graphical elements associated with the GUI 300. Examples of graphical elements associated with the GUI 300 may include, but are not limited to, buttons, sliders, dropdown lists, tabs, text boxes, and the like. The application interface 305 may also include a set of tabs enabling the user 102 to switch between different features of the application. For example, the set of tabs may allow the user 102 to switch between one or more of a “home feature,” a “readiness feature,” a “sleep feature,” or an “activity feature”, in the application running on the user device 106.

In the example of FIG. 3, biometric data collected from the user may be used to calculate a score and/or a metric (e.g., a Readiness Score) for the user. The calculated score and/or metric may be displayed to the user via the GUI 300, as shown in the application interface 305. For example, the application interface 305 may include a graphical element 310, that may display a Readiness Score 315. As noted previously, biometric data collected from a user along with application data (e.g., calendar data) may be used to provide insights and recommendations to the user. For example, the application interface 305 may include a graphical element 320, that may display a message 325, that may provide an insight to the health and wellness of the user and/or an indication of a recommended activity 330 for the user to engage in, and that may be autonomously scheduled for the user. For example, the scheduled activity 330 may be a recommendation for the user to exercise (e.g., go for a walk) between 6:00 PM and 7:00 PM.

Therefore, the GUI 300 may provide insight relevant to biometric data and application data, and their contribution to a respective health score (e.g., the Readiness Score 315) associated with the user. As such, the GUI 300 may be configured to support providing insights according to application data and biometric data. By providing personalized insights to the user, the user may experience an improvement to the health and wellness of the user (e.g., heart rate, respiratory rate, and the like).

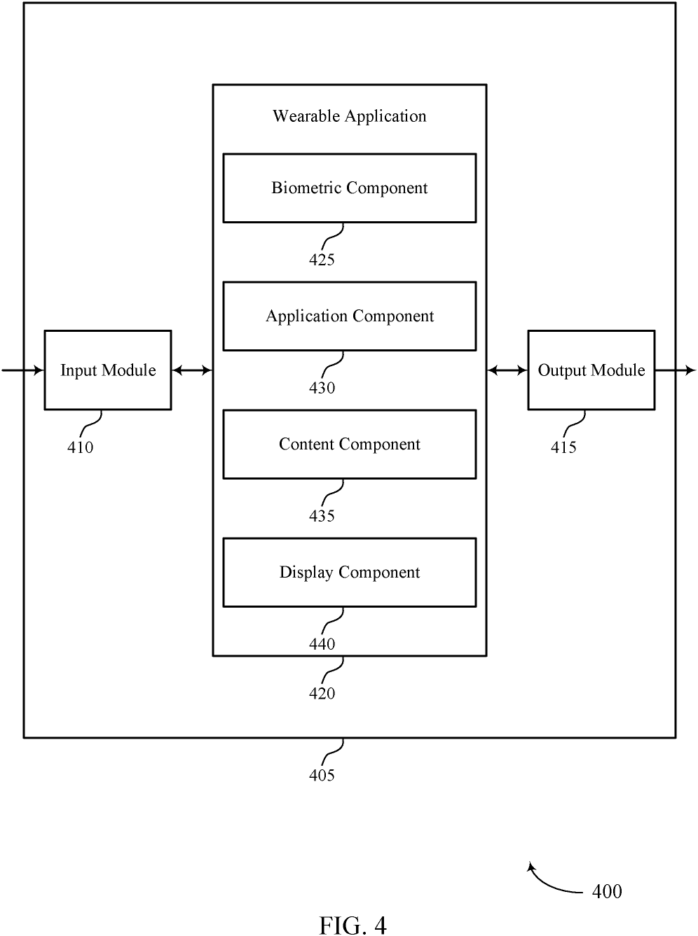

FIG. 4 shows a block diagram 400 of a device 405 that supports techniques for providing insights according to application data and biometric data in accordance with aspects of the present disclosure. The device 405 may include an input module 410, an output module 415, and a wearable application 420. The device 405 may also include a processor. Each of these components may be in communication with one another (e.g., via one or more buses).

The input module 410 may provide a means for receiving information such as packets, user data, control information, or any combination thereof associated with various information channels (e.g., control channels, data channels, information channels related to illness detection techniques). Information may be passed on to other components of the device 405. The input module 410 may utilize a single antenna or a set of multiple antennas.

The output module 415 may provide a means for transmitting signals generated by other components of the device 405. For example, the output module 415 may transmit information such as packets, user data, control information, or any combination thereof associated with various information channels (e.g., control channels, data channels, information channels related to illness detection techniques). In some examples, the output module 415 may be co-located with the input module 410 in a transceiver module. The output module 415 may utilize a single antenna or a set of multiple antennas.

For example, the wearable application 420 may include a biometric component 425, an application component 430, a content component 435, a display component 440, or any combination thereof. In some examples, the wearable application 420, or various components thereof, may be configured to perform various operations (e.g., receiving, monitoring, transmitting) using or otherwise in cooperation with the input module 410, the output module 415, or both. For example, the wearable application 420 may receive information from the input module 410, send information to the output module 415, or be integrated in combination with the input module 410, the output module 415, or both to receive information, transmit information, or perform various other operations as described herein.

The biometric component 425 may be configured as or otherwise support a means for receiving biometric data associated with a user from a wearable device. The application component 430 may be configured as or otherwise support a means for receiving application data associated with the user from a user device (e.g., the device 405), wherein the application data is indicative of a previous activity in which the user engaged or an upcoming activity in which the user is to engage. The content component 435 may be configured as or otherwise support a means for determining content for recommending to the user based at least in part on correlating the received biometric data and the received application data. The content corresponds to a respective effectiveness for regulating the received biometric data. The display component 440 may be configured as or otherwise support a means for causing a GUI of the user device (e.g., the device 405) running an application to display the content for recommending to the user based at least in part on correlating the received biometric data and the received application data.

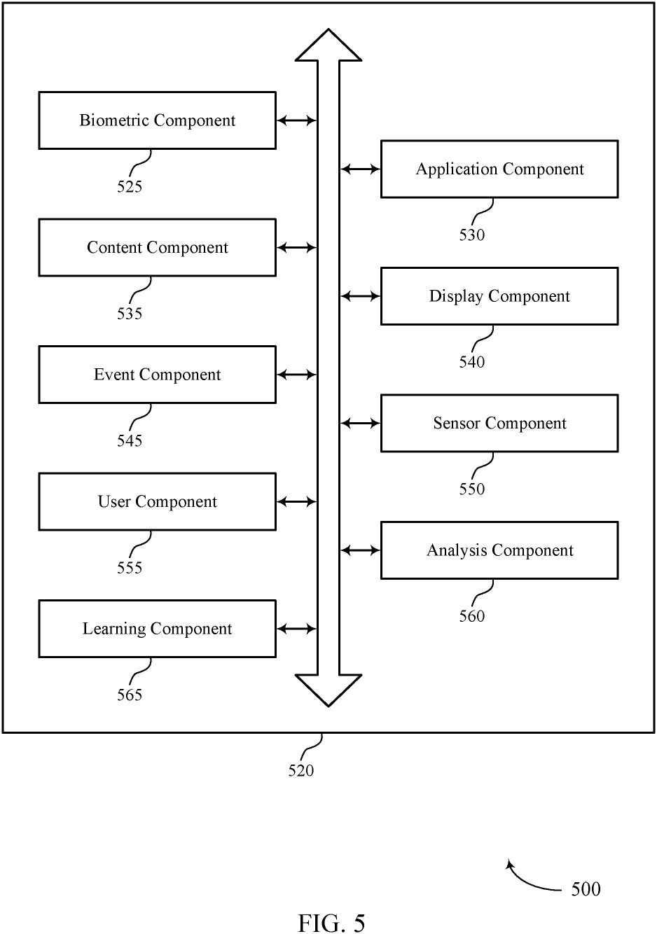

FIG. 5 shows a block diagram 500 of a wearable application 520 that supports techniques for providing insights according to application data and biometric data in accordance with aspects of the present disclosure. The wearable application 520 may be an example of aspects of a wearable application or a wearable application 420, or both, as described herein. The wearable application 520, or various components thereof, may be an example of means for performing various aspects of techniques for providing insights according to application data and biometric data as described herein. For example, the wearable application 520 may include a biometric component 525, an application component 530, a content component 535, a display component 540, an event component 545, a sensor component 550, a user component 555, an analysis component 560, a learning component 565, or any combination thereof. Each of these components may communicate, directly or indirectly, with one another (e.g., via one or more buses).

The biometric component 525 may be configured as or otherwise support a means for receiving biometric data associated with a user from a wearable device. The application component 530 may be configured as or otherwise support a means for receiving application data associated with the user from a user device. The application data is indicative of a previous activity in which the user engaged or an upcoming activity in which the user is to engage. The content component 535 may be configured as or otherwise support a means for determining content for recommending to the user based at least in part on correlating the received biometric data and the received application data, wherein the content corresponds to a respective effectiveness for regulating the received biometric data. The display component 540 may be configured as or otherwise support a means for causing a GUI of the user device running an application to display the content for recommending to the user based at least in part on correlating the received biometric data and the received application data.

In some examples, the biometric component 525 may be configured as or otherwise support a means for determining that the received biometric data satisfies a biometric threshold. In some examples, the content component 535 may be configured as or otherwise support a means for determining the content for recommending to the user further based at least in part on the received biometric data satisfying the biometric threshold. In some examples, the event component 545 may be configured as or otherwise support a means for scheduling at least one calendar event based at least in part on correlating the received biometric data and the received application data, the content comprising the at least one scheduled calendar event defined within an application associated with the wearable device and the user device. In some examples, the display component 540 may be configured as or otherwise support a means for causing the GUI of the user device running the application to display the content for recommending to the user further based at least in part on the at least one scheduled calendar event.

In some examples, the at least one scheduled calendar event comprises event information indicating an activity in which the user is to engage for regulating the received biometric data, timing information indicating a duration of the activity in which the user is to engage for regulating the received biometric data, location information indicating a location of the activity in which the user is to engage for regulating the received biometric data, or any combination thereof. In some examples, the display component 540 may be configured as or otherwise support a means for causing the GUI of the user device running the application to prompt the user for feedback based at least in part on the at least one scheduled calendar event. In some examples, the feedback comprises an acceptance of the at least one calendar event or a rejection of the at least one calendar event. In some examples, the feedback is selected from a set of feedback responses displayed via the GUI with the prompt.

In some examples, the event component 545 may be configured as or otherwise support a means for removing the at least one calendar event defined within the application associated with the wearable device and the user device based at least in part on the feedback. In some examples, the learning component 565 may be configured as or otherwise support a means for adjusting one or more parameters of a machine learning model. The machine learning model may be trained to identify the content for regulating the received biometric data based at least in part on the received application data.

In some examples, the event component 545 may be configured as or otherwise support a means for identifying at least one calendar event based at least in part on the received application data. In some examples, the content component 535 may be configured as or otherwise support a means for determining the content for recommending to the user further based at least in part on the at least one identified calendar event. The content may include a recommendation indicating one or more instructions for the user to maintain a positive or minimum Readiness Score of the user.

In some examples, the sensor component 550 may be configured as or otherwise support a means for receiving sensor data from one or both of the wearable device or the user device. The sensor data may include sound data associated with a physical environment. The content may include a recommendation indicating one or more instructions for the user to reduce a noise level during a duration to maintain a threshold Readiness Score of the user. In some examples, the content component 535 may be configured as or otherwise support a means for determining the content for recommending to the user further based at least in part on the received sensor data.

In some examples, the user component 555 may be configured as or otherwise support a means for receiving user data associated with the user from one or more applications associated with the wearable device or the user device, or any combination thereof. The one or more applications may include a lifestyle application, a social media application, a utility application, an entertainment application, a productivity application, an information outlet application, or any combination thereof. In some examples, the analysis component 560 may be configured as or otherwise support a means for correlating the received user data with the received biometric data and the received application data to determine content for recommending to the user. In some examples, the display component 540 may be configured as or otherwise support a means for causing the GUI of the user device running the application to display the content for recommending to the user further based at least in part on correlating the received user data with the received biometric data and the received application data.

In some examples, the analysis component 560 may be configured as or otherwise support a means for determining a pattern between the received biometric data and the received application data. In some examples, the analysis component 560 may be configured as or otherwise support a means for determining a recurrency of an activity over a set of occasions comprising a first occasion and a second occasion. In some examples, the biometric component 525 may be configured as or otherwise support a means for determining that at least one value of the received biometric data satisfies a threshold during a third occasion subsequent to the first occasion or the second occasion. In some examples, the content component 535 may be configured as or otherwise support a means for determining the content for recommending to the user further based at least in part on the determining that the at least one value of the received biometric data satisfies the threshold during the third occasion subsequent to the first occasion or the second occasion.

In some examples, the content comprises a user-specific notification indicating a Readiness Score or a recommendation indicating one or more instructions for the user to increase the Readiness Score of the user. In some examples, the application data comprises event information indicating an event in which the user is to engage, timing information indicating a duration of the event associated with the user, location information indicating a location of the event associated with the user, or any combination thereof. In some examples, the biometric data comprises heart rate data associated with the user, respiratory rate data associated with the user, sleep data associated with the user, activity data associated with the user, temperature data associated with the user, or any combination thereof. In some examples, the wearable device comprises a wearable ring device.

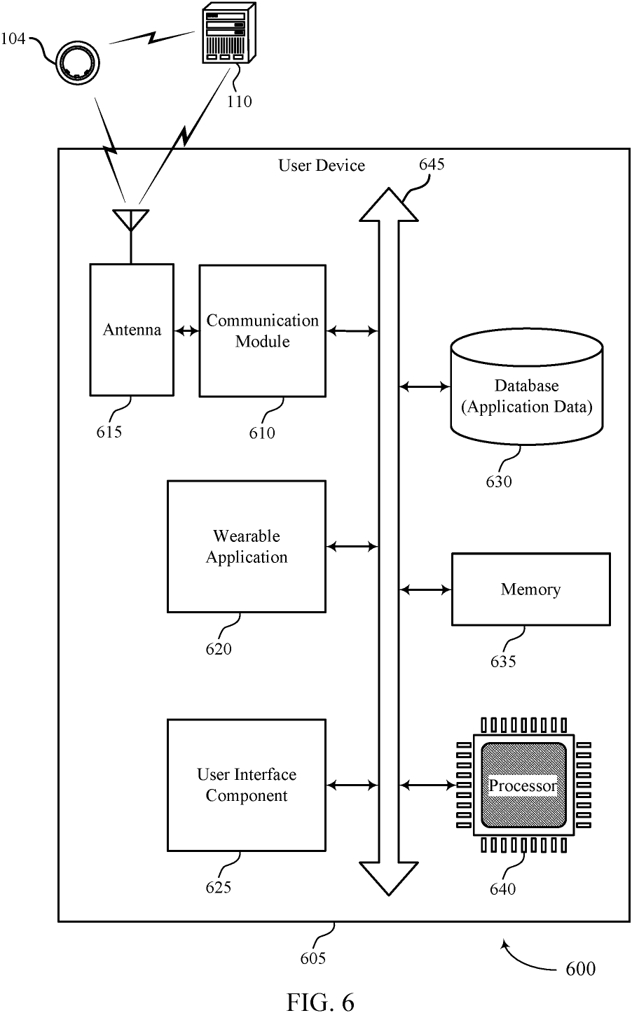

FIG. 6 shows a diagram of a system 600 including a device 605 that supports techniques for providing insights according to application data and biometric data in accordance with aspects of the present disclosure. The device 605 may be an example of or include the components of a device 405 as described herein. The device 605 may include an example of a user device 106, as described previously herein. The device 605 may include components for bi-directional communications including components for transmitting and receiving communications with a wearable device 104 and a server 110, such as a wearable application 620, a communication module 610, an antenna 615, a user interface component 625, a database (application data) 630, a memory 635, and a processor 640. These components may be in electronic communication or otherwise coupled (e.g., operatively, communicatively, functionally, electronically, electrically) via one or more buses (e.g., a bus 645).

The communication module 610 may manage input and output signals for the device 605 via the antenna 615. The communication module 610 may include an example of the communication module 220-b of the user device 106 shown and described in FIG. 2. In this regard, the communication module 610 may manage communications with the ring 104 and the server 110, as illustrated in FIG. 2. The communication module 610 may also manage peripherals not integrated into the device 605. In some cases, the communication module 610 may represent a physical connection or port to an external peripheral. In some cases, the communication module 610 may utilize an operating system such as iOS®, ANDROID®, MS-DOS®, MS-WINDOWS®, OS/2®, UNIX®, LINUX®, or another known operating system. In other cases, the communication module 610 may represent or interact with a wearable device (e.g., ring 104), modem, a keyboard, a mouse, a touchscreen, or a similar device. In some cases, the communication module 610 may be implemented as part of the processor 640. In some examples, a user may interact with the device 605 via the communication module 610, user interface component 625, or via hardware components controlled by the communication module 610.

In some cases, the device 605 may include a single antenna 615. However, in some other cases, the device 605 may have more than one antenna 615, that may be capable of concurrently transmitting or receiving multiple wireless transmissions. The communication module 610 may communicate bi-directionally, via the one or more antennas 615, wired, or wireless links as described herein. For example, the communication module 610 may represent a wireless transceiver and may communicate bi-directionally with another wireless transceiver. The communication module 610 may also include a modem to modulate the packets, to provide the modulated packets to one or more antennas 615 for transmission, and to demodulate packets received from the one or more antennas 615.

The user interface component 625 may manage data storage and processing in a database 630. In some cases, a user may interact with the user interface component 625. In other cases, the user interface component 625 may operate automatically without user interaction. The database 630 may be an example of a single database, a distributed database, multiple distributed databases, a data store, a data lake, or an emergency backup database.

The memory 635 may include RAM and ROM. The memory 635 may store computer-readable, computer-executable software including instructions that, when executed, cause the processor 640 to perform various functions described herein. In some cases, the memory 635 may contain, among other things, a BIOS that may control basic hardware or software operation such as the interaction with peripheral components or devices.

The processor 640 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 640 may be configured to operate a memory array using a memory controller. In other cases, a memory controller may be integrated into the processor 640. The processor 640 may be configured to execute computer-readable instructions stored in a memory 635 to perform various functions (e.g., functions or tasks supporting a method and system for sleep staging algorithms).

For example, the wearable application 620 may be configured as or otherwise support a means for receiving biometric data associated with a user from a wearable device. The wearable application 620 may be configured as or otherwise support a means for receiving application data associated with the user from a user device (e.g., the device 605). The application data is indicative of a previous activity in which the user engaged or an upcoming activity in which the user is to engage. The wearable application 620 may be configured as or otherwise support a means for determining content for recommending to the user based at least in part on correlating the received biometric data and the received application data. The content corresponds to a respective effectiveness for regulating the received biometric data. The wearable application 620 may be configured as or otherwise support a means for causing a GUI of the user device (e.g., the device 605) running an application to display the content for recommending to the user based at least in part on correlating the received biometric data and the received application data.

By including or configuring the wearable application 620 in accordance with examples as described herein, the device 605 may support techniques for longer battery life.

The wearable application 620 may include an application (e.g., “app”), program, software, or other component that is configured to facilitate communications with a ring 104, server 110, other user devices 106, and the like. For example, the wearable application 620 may include an application executable on a user device 106 that is configured to receive data (e.g., physiological data) from a ring 104, perform processing operations on the received data, transmit and receive data with the servers 110, and cause presentation of data to a user 102.

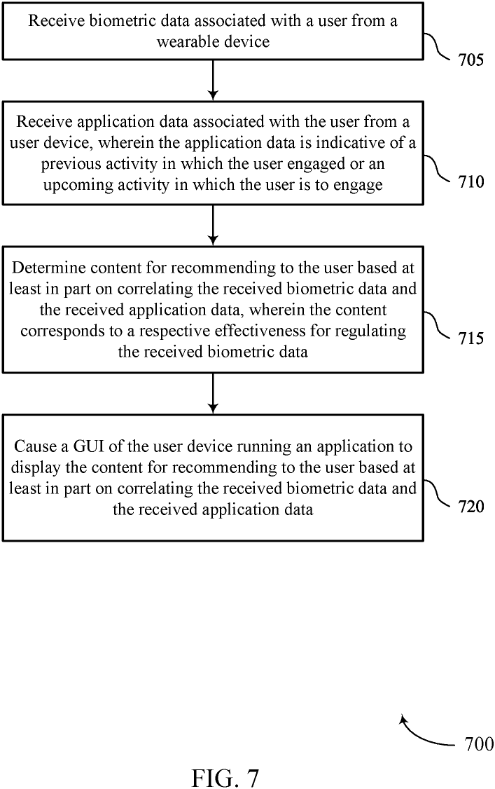

FIG. 7 shows a flowchart illustrating a method 700 that supports techniques for providing insights according to application data and biometric data in accordance with aspects of the present disclosure. The operations of the method 700 may be implemented by a user device or its components as described herein. For example, the operations of the method 700 may be performed by a user device as described with reference to FIGS. 1 through 6. In some examples, a user device may execute a set of instructions to control the functional elements of the user device to perform the described functions. Additionally or alternatively, the user device may perform aspects of the described functions using special-purpose hardware.

At 705, the method may include receiving biometric data associated with a user from a wearable device. The operations of 705 may be performed in accordance with examples as disclosed herein. In some examples, aspects of the operations of 705 may be performed by a biometric component 525 as described with reference to FIG. 5.

At 710, the method may include receiving application data associated with the user from a user device, wherein the application data is indicative of a previous activity in which the user engaged or an upcoming activity in which the user is to engage. The operations of 710 may be performed in accordance with examples as disclosed herein. In some examples, aspects of the operations of 710 may be performed by an application component 530 as described with reference to FIG. 5.

At 715, the method may include determining content for recommending to the user based at least in part on correlating the received biometric data and the received application data, wherein the content corresponds to a respective effectiveness for regulating the received biometric data. The operations of 715 may be performed in accordance with examples as disclosed herein. In some examples, aspects of the operations of 715 may be performed by a content component 535 as described with reference to FIG. 5.

At 720, the method may include causing a GUI of the user device running an application to display the content for recommending to the user based at least in part on correlating the received biometric data and the received application data. The operations of 720 may be performed in accordance with examples as disclosed herein. In some examples, aspects of the operations of 720 may be performed by a display component 540 as described with reference to FIG. 5.

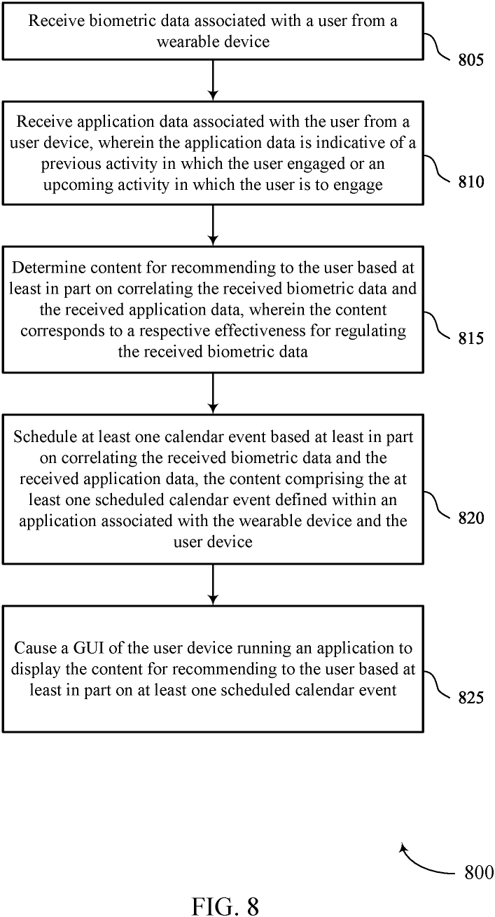

FIG. 8 shows a flowchart illustrating a method 800 that supports techniques for providing insights according to application data and biometric data in accordance with aspects of the present disclosure. The operations of the method 800 may be implemented by a user device or its components as described herein. For example, the operations of the method 800 may be performed by a user device as described with reference to FIGS. 1 through 6. In some examples, a user device may execute a set of instructions to control the functional elements of the user device to perform the described functions. Additionally or alternatively, the user device may perform aspects of the described functions using special-purpose hardware.

At 805, the method may include receiving biometric data associated with a user from a wearable device. The operations of 805 may be performed in accordance with examples as disclosed herein. In some examples, aspects of the operations of 805 may be performed by a biometric component 525 as described with reference to FIG. 5.

At 810, the method may include receiving application data associated with the user from a user device, wherein the application data is indicative of a previous activity in which the user engaged or an upcoming activity in which the user is to engage. The operations of 810 may be performed in accordance with examples as disclosed herein. In some examples, aspects of the operations of 810 may be performed by an application component 530 as described with reference to FIG. 5.

At 815, the method may include determining content for recommending to the user based at least in part on correlating the received biometric data and the received application data, wherein the content corresponds to a respective effectiveness for regulating the received biometric data. The operations of 815 may be performed in accordance with examples as disclosed herein. In some examples, aspects of the operations of 815 may be performed by a content component 535 as described with reference to FIG. 5.

At 820, the method may include scheduling at least one calendar event based at least in part on correlating the received biometric data and the received application data, the content comprising the at least one scheduled calendar event defined within an application associated with the wearable device and the user device. The operations of 820 may be performed in accordance with examples as disclosed herein. In some examples, aspects of the operations of 820 may be performed by an event component 545 as described with reference to FIG. 5.

At 825, the method may include causing a GUI of the user device running an application to display the content for recommending to the user based at least in part on at least one scheduled calendar event. The operations of 825 may be performed in accordance with examples as disclosed herein. In some examples, aspects of the operations of 825 may be performed by a display component 540 as described with reference to FIG. 5.

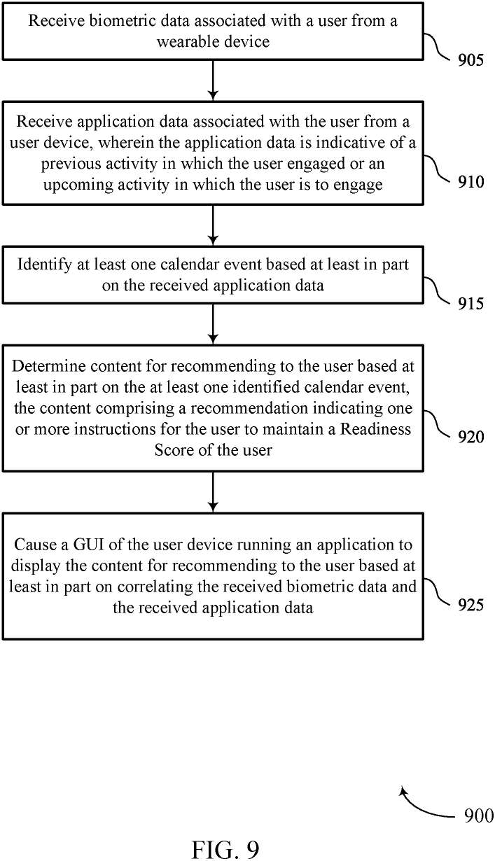

FIG. 9 shows a flowchart illustrating a method 900 that supports techniques for providing insights according to application data and biometric data in accordance with aspects of the present disclosure. The operations of the method 900 may be implemented by a user device or its components as described herein. For example, the operations of the method 900 may be performed by a user device as described with reference to FIGS. 1 through 6. In some examples, a user device may execute a set of instructions to control the functional elements of the user device to perform the described functions. Additionally or alternatively, the user device may perform aspects of the described functions using special-purpose hardware.

At 905, the method may include receiving biometric data associated with a user from a wearable device. The operations of 905 may be performed in accordance with examples as disclosed herein. In some examples, aspects of the operations of 905 may be performed by a biometric component 525 as described with reference to FIG. 5.

At 910, the method may include receiving application data associated with the user from a user device, wherein the application data is indicative of a previous activity in which the user engaged or an upcoming activity in which the user is to engage. The operations of 910 may be performed in accordance with examples as disclosed herein. In some examples, aspects of the operations of 910 may be performed by an application component 530 as described with reference to FIG. 5.

At 915, the method may include identifying at least one calendar event based at least in part on the received application data. The operations of 915 may be performed in accordance with examples as disclosed herein. In some examples, aspects of the operations of 915 may be performed by an event component 545 as described with reference to FIG. 5.

At 920, the method may include determining content for recommending to the user based at least in part on the at least one identified calendar event, the content comprising a recommendation indicating one or more instructions for the user to maintain a Readiness Score of the user. The operations of 920 may be performed in accordance with examples as disclosed herein. In some examples, aspects of the operations of 920 may be performed by a content component 535 as described with reference to FIG. 5.

At 925, the method may include causing a GUI of the user device running an application to display the content for recommending to the user based at least in part on correlating the received biometric data and the received application data. The operations of 925 may be performed in accordance with examples as disclosed herein. In some examples, aspects of the operations of 925 may be performed by a display component 540 as described with reference to FIG. 5.

It should be noted that the methods described above describe possible implementations, and that the operations and the steps may be rearranged or otherwise modified and that other implementations are possible. Furthermore, aspects from two or more of the methods may be combined.

The following provides an overview of the present disclosure:

A method is described. The method may include receiving biometric data associated with a user from a wearable device, receiving application data associated with the user from a user device, wherein the application data is indicative of a previous activity in which the user engaged or an upcoming activity in which the user is to engage, determining content for recommending to the user based at least in part on correlating the received biometric data and the received application data, wherein the content corresponds to a respective effectiveness for regulating the received biometric data, and causing a GUI of the user device running an application to display the content for recommending to the user based at least in part on correlating the received biometric data and the received application data.

An apparatus is described. The apparatus may include a processor, memory coupled with the processor, and instructions stored in the memory. The instructions may be executable by the processor to cause the apparatus to receive biometric data associated with a user from a wearable device, receive application data associated with the user from the apparatus, wherein the application data is indicative of a previous activity in which the user engaged or an upcoming activity in which the user is to engage, determine content for recommending to the user based at least in part on correlating the received biometric data and the received application data, wherein the content corresponds to a respective effectiveness for regulating the received biometric data, and cause a GUI of the apparatus running an application to display the content for recommending to the user based at least in part on correlating the received biometric data and the received application data.

Another apparatus is described. The apparatus may include means for receiving biometric data associated with a user from a wearable device, means for receiving application data associated with the user from the apparatus, wherein the application data is indicative of a previous activity in which the user engaged or an upcoming activity in which the user is to engage, means for determining content for recommending to the user based at least in part on correlating the received biometric data and the received application data, wherein the content corresponds to a respective effectiveness for regulating the received biometric data, and means for causing a GUI of the apparatus running an application to display the content for recommending to the user based at least in part on correlating the received biometric data and the received application data.

A non-transitory computer-readable medium storing code is described. The code may include instructions executable by a processor to receive biometric data associated with a user from a wearable device, receive application data associated with the user from a user device, wherein the application data is indicative of a previous activity in which the user engaged or an upcoming activity in which the user is to engage, determine content for recommending to the user based at least in part on correlating the received biometric data and the received application data, wherein the content corresponds to a respective effectiveness for regulating the received biometric data, and cause a GUI of the user device running an application to display the content for recommending to the user based at least in part on correlating the received biometric data and the received application data.

Some examples of the method, apparatuses, and non-transitory computer-readable medium described herein may further include operations, features, means, or instructions for determining that the received biometric data satisfies a biometric threshold, wherein determining the content for recommending to the user may be further based at least in part on the received biometric data satisfying the biometric threshold.

Some examples of the method, apparatuses, and non-transitory computer-readable medium described herein may further include operations, features, means, or instructions for scheduling at least one calendar event based at least in part on correlating the received biometric data and the received application data, the content comprising the at least one scheduled calendar event defined within an application associated with the wearable device and the user device, wherein causing the GUI of the user device running the application to display the content for recommending to the user may be further based at least in part on the at least one scheduled calendar event.

In some examples of the method, apparatuses, and non-transitory computer-readable medium described herein, the at least one scheduled calendar event comprises event information indicating an activity in which the user is to engage for regulating the received biometric data, timing information indicating a duration of the activity in which the user is to engage for regulating the received biometric data, location information indicating a location of the activity in which the user is to engage for regulating the received biometric data, or any combination thereof.

Some examples of the method, apparatuses, and non-transitory computer-readable medium described herein may further include operations, features, means, or instructions for causing the GUI of the user device running the application to prompt the user for feedback based at least in part on the at least one scheduled calendar event.

In some examples of the method, apparatuses, and non-transitory computer-readable medium described herein, the feedback comprises an acceptance of the at least one calendar event or a rejection of the at least one calendar event and the feedback may be selected from a set of feedback responses displayed via the GUI with the prompt.

Some examples of the method, apparatuses, and non-transitory computer-readable medium described herein may further include operations, features, means, or instructions for removing the at least one calendar event defined within the application associated with the wearable device and the user device based at least in part on the feedback and adjusting one or more parameters of a machine learning model, wherein the machine learning model may be trained to identify the content for regulating the received biometric data based at least in part on the received application data.

Some examples of the method, apparatuses, and non-transitory computer-readable medium described herein may further include operations, features, means, or instructions for identifying at least one calendar event based at least in part on the received application data and wherein determining the content for recommending to the user may be further based at least in part on the at least one identified calendar event, the content comprising a recommendation indicating one or more instructions for the user to maintain a threshold Readiness Score of the user.

Some examples of the method, apparatuses, and non-transitory computer-readable medium described herein may further include operations, features, means, or instructions for receiving sensor data from one or both of the wearable device or the user device, the sensor data comprising sound data associated with a physical environment, the content comprising a recommendation indicating one or more instructions for the user to reduce a noise level during a duration to maintain a threshold Readiness Score of the user and wherein determining the content for recommending to the user may be further based at least in part on the received sensor data.

Some examples of the method, apparatuses, and non-transitory computer-readable medium described herein may further include operations, features, means, or instructions for receiving user data associated with the user from one or more applications associated with the wearable device or the user device, or any combination thereof, the one or more applications comprising a lifestyle application, a social media application, a utility application, an entertainment application, a productivity application, an information outlet application, or any combination thereof, correlating the received user data with the received biometric data and the received application data to determine content for recommending to the user, and wherein causing the GUI of the user device running the application to display the content for recommending to the user may be further based at least in part on correlating the received user data with the received biometric data and the received application data.

Some examples of the method, apparatuses, and non-transitory computer-readable medium described herein may further include operations, features, means, or instructions for determining a pattern between the received biometric data and the received application data.

Some examples of the method, apparatuses, and non-transitory computer-readable medium described herein may further include operations, features, means, or instructions for determining a recurrency of an activity over a set of occasions comprising a first occasion and a second occasion, determining that at least one value of the received biometric data satisfies a threshold during a third occasion subsequent to the first occasion or the second occasion, wherein determining the content for recommending to the user may be further based at least in part on the determining that the at least one value of the received biometric data satisfies the threshold during the third occasion subsequent to the first occasion or the second occasion.

In some examples of the method, apparatuses, and non-transitory computer-readable medium described herein, the content comprises a user-specific notification indicating a Readiness Score or a recommendation indicating one or more instructions for the user to increase the Readiness Score of the user.

In some examples of the method, apparatuses, and non-transitory computer-readable medium described herein, the application data comprises event information indicating an event in which the user is to engage, timing information indicating a duration of the event associated with the user, location information indicating a location of the event associated with the user, temperature data associated with the user, or any combination thereof.

In some examples of the method, apparatuses, and non-transitory computer-readable medium described herein, the biometric data comprises heart rate data associated with the user, respiratory rate data associated with the user, sleep data associated with the user, activity data associated with the user, or any combination thereof.

In some examples of the method, apparatuses, and non-transitory computer-readable medium described herein, the wearable device comprises a wearable ring device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method comprising:
receiving biometric data associated with a user from a wearable device;
receiving application data associated with the user from a user device, wherein the application data is indicative of a previous activity in which the user engaged or an upcoming activity in which the user is to engage;
determining content for recommending to the user based at least in part on correlating the received biometric data and the received application data, wherein the content corresponds to a respective effectiveness for regulating the received biometric data; and
causing a graphical user interface of the user device running an application to display the content for recommending to the user based at least in part on correlating the received biometric data and the received application data.

2. The method of claim 1, further comprising:
determining that the received biometric data satisfies a biometric threshold,
wherein determining the content for recommending to the user is further based at least in part on the received biometric data satisfying the biometric threshold.

3. The method of claim 1, further comprising:
scheduling at least one calendar event based at least in part on correlating the received biometric data and the received application data, the content comprising the at least one scheduled calendar event defined within an application associated with the wearable device and the user device,
wherein causing the graphical user interface of the user device running the application to display the content for recommending to the user is further based at least in part on the at least one scheduled calendar event.

4. The method of claim 3, wherein the at least one scheduled calendar event comprises event information indicating an activity in which the user is to engage for regulating the received biometric data, timing information indicating a duration of the activity in which the user is to engage for regulating the received biometric data, location information indicating a location of the activity in which the user is to engage for regulating the received biometric data, or any combination thereof.

5. The method of claim 3, further comprising:
causing the graphical user interface of the user device running the application to prompt the user for feedback based at least in part on the at least one scheduled calendar event.

6. The method of claim 5, wherein the feedback comprises an acceptance of the at least one calendar event or a rejection of the at least one calendar event, and the feedback is selected from a set of feedback responses displayed via the graphical user interface with the prompt.

7. The method of claim 5, further comprising:
removing the at least one calendar event defined within the application associated with the wearable device and the user device based at least in part on the feedback; and
adjusting one or more parameters of a machine learning model, wherein the machine learning model is trained to identify the content for regulating the received biometric data based at least in part on the received application data.

8. The method of claim 1, further comprising:
identifying at least one calendar event based at least in part on the received application data,
wherein determining the content for recommending to the user is further based at least in part on the at least one identified calendar event, the content comprising a recommendation indicating one or more instructions for the user to maintain a threshold Readiness Score of the user.

9. The method of claim 1, further comprising:
receiving sensor data from one or both of the wearable device or the user device, the sensor data comprising sound data associated with a physical environment, the content comprising a recommendation indicating one or more instructions for the user to reduce a noise level during a duration to maintain a threshold Readiness Score of the user,
wherein determining the content for recommending to the user is further based at least in part on the received sensor data.

10. The method of claim 1, further comprising:
receiving user data associated with the user from one or more applications associated with the wearable device or the user device, or any combination thereof, the one or more applications comprising a lifestyle application, a social media application, a utility application, an entertainment application, a productivity application, an information outlet application, or any combination thereof; and
correlating the received user data with the received biometric data and the received application data to determine content for recommending to the user,
wherein causing the graphical user interface of the user device running the application to display the content for recommending to the user is further based at least in part on correlating the received user data with the received biometric data and the received application data.

11. The method of claim 1, further comprising:
determining a pattern between the received biometric data and the received application data.

12. The method of claim 11, further comprising:
determining a recurrency of an activity over a set of occasions comprising a first occasion and a second occasion; and
determining that at least one value of the received biometric data satisfies a threshold during a third occasion subsequent to the first occasion or the second occasion,
wherein determining the content for recommending to the user is further based at least in part on the determining that the at least one value of the received biometric data satisfies the threshold during the third occasion subsequent to the first occasion or the second occasion.

13. The method of claim 12, wherein the content comprises a user-specific notification indicating a Readiness Score or a recommendation indicating one or more instructions for the user to increase the Readiness Score of the user.

14. The method of claim 1, wherein the application data comprises event information indicating an event in which the user is to engage, timing information indicating a duration of the event associated with the user, location information indicating a location of the event associated with the user, temperature data associated with the user, or any combination thereof.

15. The method of claim 1, wherein the biometric data comprises heart rate data associated with the user, respiratory rate data associated with the user, sleep data associated with the user, activity data associated with the user, or any combination thereof.

16. The method of claim 1, wherein the wearable device comprises a wearable ring device.

17. An apparatus, comprising:
a processor;
memory coupled with the processor; and
instructions stored in the memory and executable by the processor to cause the apparatus to:
receive biometric data associated with a user from a wearable device;
receive application data associated with the user from the apparatus, wherein the application data is indicative of a previous activity in which the user engaged or an upcoming activity in which the user is to engage;
determine content for recommending to the user based at least in part on correlating the received biometric data and the received application data, wherein the content corresponds to a respective effectiveness for regulating the received biometric data; and
cause a graphical user interface of the apparatus running an application to display the content for recommending to the user based at least in part on correlating the received biometric data and the received application data.

18. The apparatus of claim 17, wherein the instructions are further executable by the processor to cause the apparatus to:
determine that the received biometric data satisfies a biometric threshold,
wherein the instructions to determine the content for recommending to the user are further executable by the processor based at least in part on the received biometric data satisfying the biometric threshold.

19. The apparatus of claim 17, wherein the instructions are further executable by the processor to cause the apparatus to:
schedule at least one calendar event based at least in part on correlating the received biometric data and the received application data, the content comprising the at least one scheduled calendar event defined within an application associated with the wearable device and the user device,
wherein the instructions to cause the graphical user interface of the apparatus running the application to display the content for recommending to the user are further executable by the processor based at least in part on the at least one scheduled calendar event.

20. A non-transitory computer-readable medium storing code, the code comprising instructions executable by a processor to:
receive biometric data associated with a user from a wearable device;
receive application data associated with the user from a user device, wherein the application data is indicative of a previous activity in which the user engaged or an upcoming activity in which the user is to engage;
determine content for recommending to the user based at least in part on correlating the received biometric data and the received application data, wherein the content corresponds to a respective effectiveness for regulating the received biometric data; and
cause a graphical user interface of the user device running an application to display the content for recommending to the user based at least in part on correlating the received biometric data and the received application data.
